(19) United States
(12) Patent Application Publication
(10) Pub. No.: US 2023/0369996 A1
(43) Pub. Date: Nov. 16, 2023

(54) Implantable Power Generator

(71) Applicant: Timm A. Vanderelli, Ligonier, PA (US)

(72) Inventor: Timm A. Vanderelli, Ligonier, PA (US)

(21) Appl. No.: 17/744,680

(22) Filed: May 15, 2022

(51) Int. Cl. H02N 11/00; H02K 35/02

(52) U.S. Cl. CPC H02N 11/002; H02K 35/02; A61N 1/3785

(57) ABSTRACT

My invention is a power generator, of which the preferred embodiment is small enough to be implanted into the human body, shaped to fit into the area below the lungs, affixed to the interior body cavity and activated by the rhythmic movement of the diaphragm. A human diaphragm will move up to fifty-millimeters during the involuntary breathing process while a body is resting, in which my implantable power generator will output a stream of pulsed electric current with each inhale and another stream of pulsed electric current while exhaling during the body's minimum breathing period. My implantable power generator has sufficient output energy to supply a pacemaker, implantable pulse generator (IPG) or other medical implants that consume a constant supply of power without using internal batteries that would require repeated surgeries due to constant replacement or external battery packs that require passing wires through the skin and into the body.

### CROSS-REFERENCE TO RELATED APPLICATIONS

This application claims the benefit of provisional patent application Ser. No. US 42/923869 filed Jun. 8, 2021 by the present inventor.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not applicable

### TECHNICAL FIELD

The present invention relates to electric generators that utilize linear kinetic motion as a source of energy and specifically, energy obtained from a body's moving muscle tissues.

### PRIOR ART

The following is tabulation of some prior art that presently appears relevant:

U.S. Patents

| Patent Number | Issue Date | Patentee |
|---|---|---|
| U.S. Pat. No. 3,968,387 | Jul. 6, 1976 | Scarff, David |
| U.S. Pat. No. 6,020,653 | Feb. 1, 2000 | Woodbridge, David |
| U.S. Pat. No. 8,766,493 | Jul. 1, 2014 | Hunter, Ian. |
| U.S. Pat. No. 10,038,349 | Jul. 31, 2018 | Long, J. D |

The patents listed above are distinguishable from the present invention by the following non-inclusive comparisons.

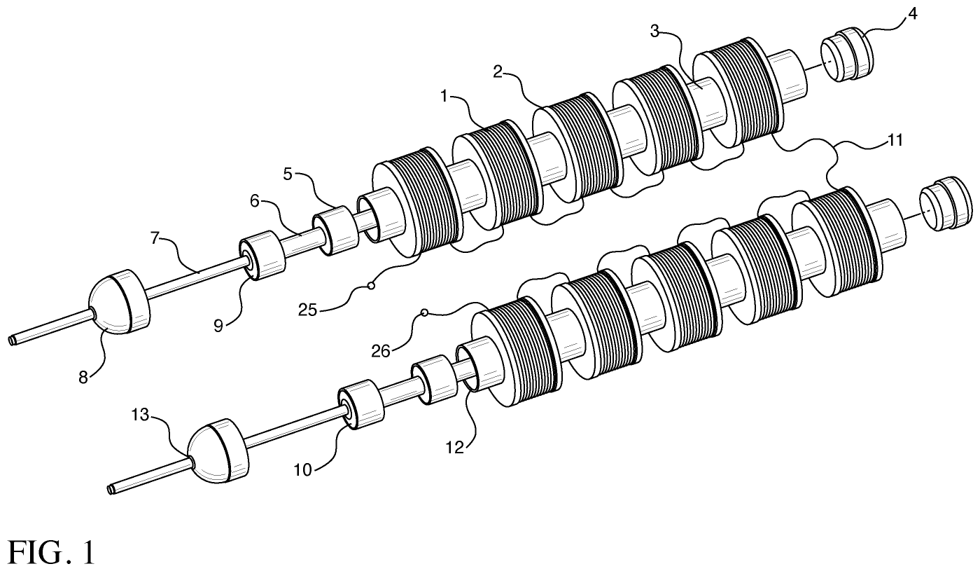

U.S. Pat. No. 3,968,387—Teaches two electromagnets surrounding ferrous alloy cores axially oriented on either side of a bar magnet centrally located within generating coil, as shown in his FIG. 1.

I teach a column of ring magnets with their magnetic poles axially aligned and located inside an inductor as shown in my FIG. 1.

U.S. Pat. No. 6,020,653—Utilizes a rotating drum, affixed to a cable for coupling kinetic energy to a moving generator support tube over fixed permanent magnets, as shown in his FIG. 1.

I teach a linear moving, non-metallic rod without a rotating drum to couple kinetic energy to moving magnets inside of inductors, as shown in my FIG. 1.

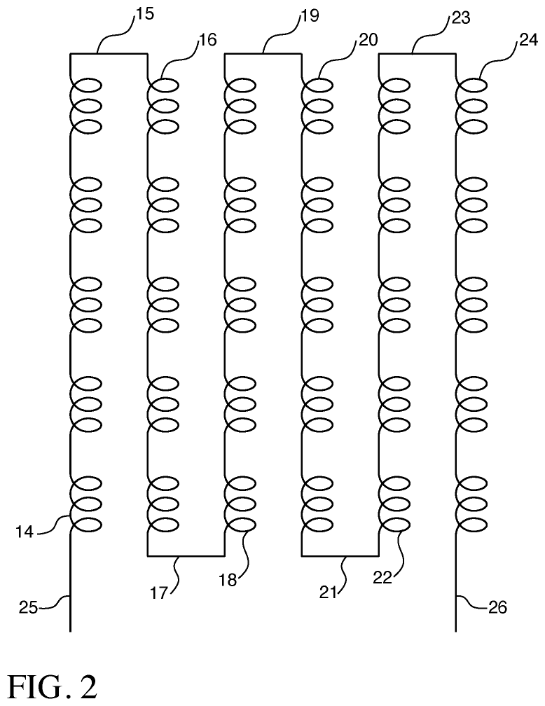

U.S. Pat. No. 8,766,493—Teaches alternating magnetic polarity alignment of multiple linearly aligned magnets as shown in his FIGS. 2A, 2B and 2C. I teach same magnetic polarity alignment of a column of linearly aligned magnets as shown in my FIG. 1.

U.S. Ser. No. 10/038,349—Teaches alternating magnetic polarity alignment of multiple linearly arranged magnets, including two opposing stationary magnets on either end, as shown in his FIG. 1. I teach same polarity magnetic alignment of multiple linearly arranged magnets in a linear column without utilizing additional, opposing stationary magnets as seen in my FIG. 1.

Abstract

My invention is a power generator, of which the preferred embodiment is small enough to be implanted into the human body, shaped to fit into the area below the lungs, affixed to the interior body cavity and activated by the rhythmic movement of the diaphragm. A human diaphragm will move up to fifty-millimeters during the involuntary breathing process while a body is resting, in which my implantable power generator will output a stream of pulsed electric current with each inhale and another stream of pulsed electric current while exhaling during the body's minimum breathing period. My implantable power generator has sufficient output energy to supply a pacemaker, implantable pulse generator (IPG) or other medical implants that consume a constant supply of power without using internal batteries that would require repeated surgeries due to constant replacement or external battery packs that require passing wires through the skin and into the body.

### TECHNICAL FIELD

The present invention relates to electric power generators, specifically a small, implantable generator designed to output useful electric currents created by the body's continuous and cyclic muscle movements.

### BACKGROUND

A number of methods have been tried in order to generate electric currents from the human body. An internal method is the use of micro-generators within the arteries creating “vascular turbines” while another related method harvests a few microwatts from the beating of the heart. This micro-turbine method produces very little current and may be usable for implants that require very low amounts of power. Another external method for producing currents can be obtained from body heat while another relies on piezo electric harvesting from muscle motion, including walking. All of these methods are experimental, not in common practice and produce little usable energy. A widely accepted and commonly used method for powering implants from external chargers is through inductive coupling, whereby high frequency waves are transmitted into a first inductor having a small magnet located in the center on the outside of the skin for energy transfer and support via a second inductor implanted below the skin, which is designed to be in electromagnetic resonance with said first inductor. First inductor is held in position on the outside of the skin by the magnetic attraction obtained of a small ferrous plate located in said second inductor's center. Received radio frequencies from said second inductor are then rectified, filtered and regulated for powering implants. Inductive coupling is preferred for implants that require very little power to operate due to complications arising from body tissue heating resulting from high levels of radio frequency waves passing through the skin. The Federal Communications Commission has imposed limits of 4 mw/cm for the general population in relation to this concern for human health. In comparison, a major drawback of wired, human-powered producing methods for implants is they rely on wires passing through the skin and pose just as much of a health problem as external battery packs do by way of infection, disease, inconvenience and discomfort.

### SUMMARY AND ADVANTAGES

The implementation of my invention will greatly reduce the risks of infection and disease by halting the need for wires passing through the skin or the replacement of internal batteries that require repeated surgeries, also eliminating the associated inconvenience and discomfort of an external power source. Once my implantable power generator is installed into the body, no further maintenance should be required, thus eliminating the use of internal replaceable batteries.

### DESCRIPTION OF THE DRAWINGS

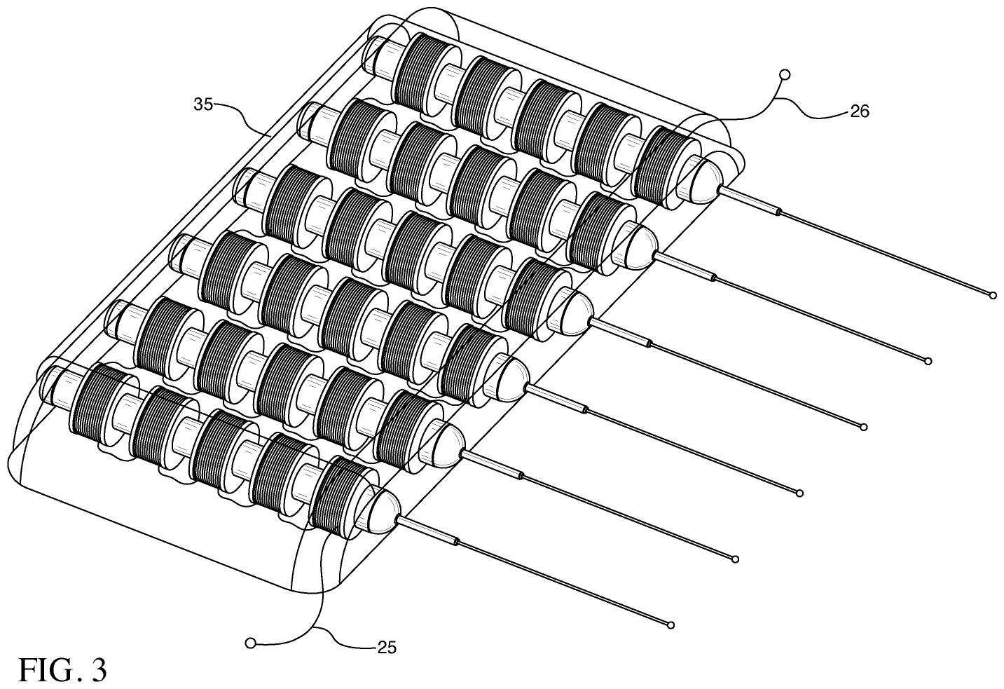

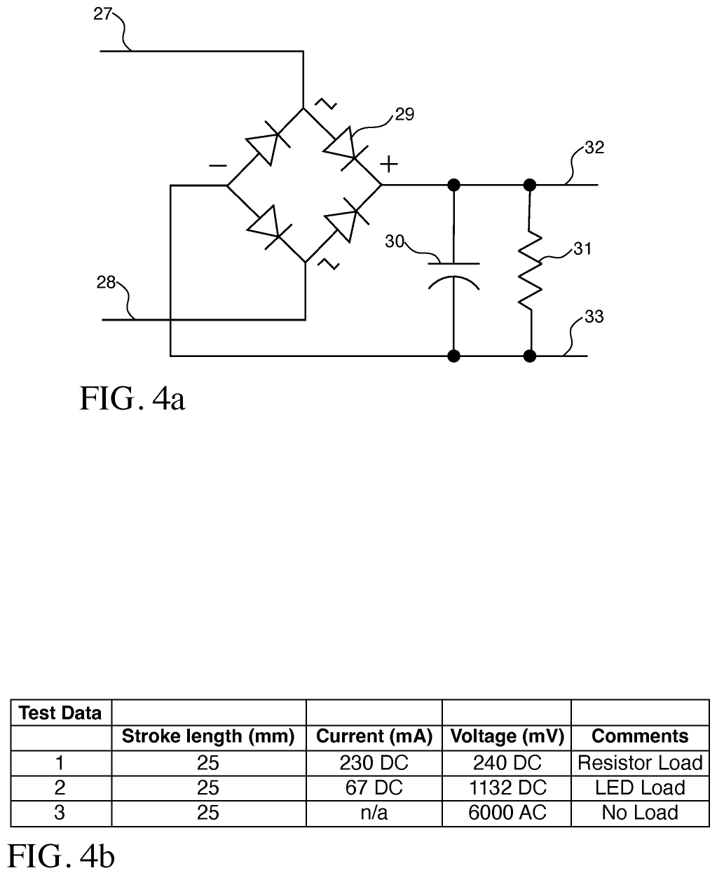

Referring to FIG. 1, a first and second group of five small inductors are each wound in a clockwise direction, using three-hundred turns of forty-gauge enameled copper wire 1 and spaced apart evenly on two flexible plastic, hollow rods 3. Said inductors are held in position and affixed to the outer side of said hollow rods by twenty thin disks 2 which provide horizontal support for said wound wires. Plastic first end-cap 4 press fits into first end of said hollow rod 3 to prevent debris from entering said first and second hollow rods. Ten ring magnets 5 are affixed to a first flexible non-metallic rod 6 with an adhesive and aligned with the first of five magnetic north 9 all facing in the same direction. Within the second group of five small inductors a second five ring magnets are aligned with their magnetic souths facing in the same direction 10. A second flexible non-metallic thin rod 7 is affixed to top end of each magnet assembly 9. Second plastic end-cap 8 passes over said second thin rod 7 by way of through-hole 13 and is press-fitted over said second end of hollow rod 3 and affixed in place with an adhesive. FIG. 2 shows a schematic diagram of the interconnections of said inductors 1 and output wires 25, 26 of the preferred embodiment that was constructed using a six-column matrix as shown in FIG. 3. Referring to FIG. 3, the preferred embodiment utilizes six said magnet assemblies arranged in three groups of two out of phase columns 9, 10 as shown in FIG. 1. Additionally, six groups of five said inductors 1 mounted on said hollow rod 3 are electrically connected in series and horizontally juxtaposed to one another, forming a thirty-cell matrix of inductors and magnets. A schematic diagram of a typical alternating current to direct current power supply, consisting of a diode bridge rectifier 29, electrolytic capacitor 30 and load resistor 31 was used to gather test data as shown in FIG. 4a. Test data gathered from said preferred embodiment is shown in FIG. 4b.

### DESCRIPTION OF THE INVENTION

A preferred embodiment of my invention is shown in FIG. 3. It consists of six columns of five ten-milli-Henry inductors spaced four-millimeters apart and wired in series as shown in FIG. 2, the schematic diagram indicating how the first five inductors 14 connect to second five inductors 16 via out of phase connection 15, with the pattern repeating as shown in 16, 17, 18, 19, 21, 20, 22, 23 and 24, with alternating output currents obtained from wires 25 and 26. Referring to FIG. 1, six ring magnet assemblies 5 fabricated with five eight-millimeter diameter by four millimeter-thick ring magnets, magnetized through their thickness with a magnetic pole B-field strength of one-thousand Gauss, spaced eight-millimeters apart and affixed to six individual, three-millimeter diameter said first rod 6 with an adhesive, each located inside columns of six said hollow rods 3, their dimensions fifty-millimeters in length, 8.25 millimeters inner diameter with a wall thickness of 0.25 millimeters, spaced eight-millimeters apart, where a matrix of six said magnet columns 5 are free to slide in and out of said hollow rods 3 with a stroke of up to forty-five-millimeters by way of six one-millimeter diameter said second thin rod 7, one end affixed to each said magnet column 5 with the opposite ends of said thin rod 7 attached to the body's diaphragm. Each alternative, successive column of said magnet columns 9, 10 have their magnetic polarities reversed one-hundred-eighty degrees to compensate for the phase inversion of each adjacent column of said inductors 11. The entire inductor-magnet matrix assembly is encased within a suitably-sized, flexible silicon housing 35 and attached to the inside of the body's interior wall in a location and by a method determined during the research and development stage by medical professionals. Movement of the body's diaphragm is responsible for movement of said magnet columns, via six said thin rods 7 of varying length between ten-millimeters and one-hundred-millimeters, depending on their individual location of attachment to the body's arched-shaped diaphragm, and their synchronicity is to be determined by the surgeon preforming the installation of my invention. Synchronized motion of said magnet columns 5 induce currents into said inductors 1, creating potential energy. Referencing FIG. 3, the thirty said inductors 1, wired in series result in a total inductance measuring three-hundred-milliHenries; having a total direct current resistance of thirty-Ohms, arranged into a matrix and encased in a soft, flexible silicon housing one-hundred-millimeters long, fifteen-millimeters thick and fifty-millimeters long. Five magnets per said column creates five pulses of approximately five-volts each per inhale stroke and another five, approximately five-volt pulses each per exhale stroke from the body's breathing cycle. Useful energy is obtained from the thirty, series-connected inductor matrix as pulsed alternating currents that can be full-wave rectified, filtered and stored in a capacitor and regulated using common power supply circuitry for supplying regulated power to medical implants in the human body. Those skilled in the art can see my preferred embodiment is not limited only by connection to the body's diaphragm, but any other moving muscle in the body can activate said invention. Nor is there a limit of six fifty-millimeter columns of said inductor-magnet matrix, nor limits of physical dimensions but said power generator's matrix construction can be scaled to meet the requirements of the chosen device requiring power and can be connected to any moving muscle for receiving kinetic energy.

Test Data

Referring to FIG. 4b, output tests were performed on the preferred embodiment of my implantable power generator and the results were observed and recorded. In a first test, the preferred embodiment shown in FIG. 3 was connected to a full-wave germanium diode bridge rectifier 29 shown in schematic diagram FIG. 4a, by way of inputs 27 and 28 from said inductor output connections 25 and 26 as shown in FIGS. 1, 2 and 3. Referring back to FIG. 4a, the addition of a one-microfarad electrolytic capacitor 30 was connected in parallel with said rectifier's positive and negative outputs with a one-thousand Ohm carbon resistor connected in parallel 31 with said rectifier and capacitor and utilized as a load as shown in FIG. 4a, to create a test apparatus for gathering voltage and current data. Referring to FIG. 1, a single, one-second duration, twenty-five-millimeter-long stroke of inward motion of said magnet columns 5 generated power as shown in FIG. 4b from output leads 32, 33 as indicated in test data reference 1, FIG. 4b, which resulted in a loaded output voltage of two-hundred-forty milliVolts DC with currents of two-hundred-thirty-milliAmperes, producing fifty-three-milliWatts of usable power obtained from a single twenty-five-millimeter stroke. A second test is shown utilizing a typical red light emitting diode (LED) which was substituted for said load resistor 31 and connected directly across the outputs 32, 33 of said test apparatus, and during each inward and each outward stroke, the measured voltage across said LED was 1.13 volts with measured DC current through said LED of sixty-seven milliAmperes as indicated in test data 2, FIG. 4b. It was observed that said LED flashed brightly five times with each one-second inward stroke and flashed again five times with each one-second outward stroke. A third open circuit test (un-loaded) of the preferred embodiment is shown in test data 3, which produced five pulses of six-volts AC, peak-to-peak as indicated in FIG. 4b, at output leads 25, 26 as shown in FIGS. 2, 3, by moving said magnet columns 5 as shown in FIG. 1, over a distance of twenty-five millimeters in one direction during one second.

1. I claim a power generator comprising,
a. one or more columns of four or more inductors adjacent to and evenly spaced apart, electrically wired in series, juxtaposed to one another, with each successive column electrically wired out of phase with one another;
b. one or more actuator magnet assemblies, each consisting of a linear alignment of four or more magnets, their magnetic polarity aligned in the same direction, affixed to a flexible, non-metallic rod, evenly spaced apart,
c. with each adjacent column having a magnetic polarity in opposition of 180 degrees;
d. a flexible housing which holds said inductors and actuator magnet columns, preferably made of silicon or any other soft, flexible material;
e. at least one flexible, non-metallic rod with one end affixed to one end of said magnets and the opposite end for connection to a fixed location on a movable object.
